(19) United States
(12) Patent Application Publication
(10) Pub. No.: US 2007/0243151 A1
(43) Pub. Date: Oct. 18, 2007

(54) Nonaqueous Gel Compositions

(76) Inventor: Lin Lu Healy, Houston, TX (US)

Correspondence Address: ARTHUR M DULA;LAW OFFICE OF ARTHUR M DULA, 3106 BEAUCHAMP STREET, HOUSTON, TX, 77009, US

(21) Appl. No.: 11/279,868

(22) Filed: Apr. 14, 2006

(51) Int. Cl. A61K 8/81; A61K 8/37

(52) U.S. Cl. 424/64

(57) ABSTRACT

The present invention is a non-aqueous cosmetic gel composition of from about 47% to 62% by weight anhydrous gel, from about 17% to about 32% by weight polymer, from about 8% to about 22% by weight ester, and from about 0.001% to about 15% by weight gelling agent wherein the inclusion of the gelling agents provides a means for delivery of the emollients.

### FIELD OF THE INVENTION

The invention relates to a non-aqueous gel composition for cosmetic use. Specifically, the invention relates to a clear or semi-clear lipstick product utilizing thermoplastic copolymers, hydrocarbon oils, esters and amino acid based gelling agents. The stick is translucent, stiff, and gives good pay-off. The stick may be used as a delivery vehicle for emollients.

### BACKGROUND OF THE INVENTION

Clear rigid gels are desirable products in the cosmetic and personal care industry. While there are many prior art methods to make clear aqueous gels, there are few methods of making clear non-aqueous gel compositions. Aqueous gels are clear and strong, but they dry out and crack as the aqueous component volatilizes. Prior art clear non-aqueous gels are often very soft, and they exhibit such undesirable properties as synerisis and haziness. The prior art non-aqueous gels that are clear and rigid, such as gels made with Versagel® R for cosmetic use, exhibit high elasticity and low “pay-off” (a term used to describe both the amount of product, such as lipstick, and the way the product distributes on the user's skin). These qualities make all prior art non-aqueous gels known to the present inventor inadequate where a non-aqueous gel is desired that possesses the qualities of clarity, rigidity, non-elasticity, and high pay-off.

U.S. Pat. No. 5,221,534 teaches a health and beauty aid composition comprising diblock copolymers such as a styrene butadiene copolymer, along with an organic ester solvent such as C₁₂₋₁₅ alkyl benzoate.

U.S. Pat. No. 6,468,510 teaches a transparent lipstick composition comprising an N-acyl glutamic acid diamide (sold by Ajinomoto Co., Inc., under the trade designation GP-1) as an oil-gelatinizing agent. However, the present invention combines GP-1 with an ester compound (C12-C15 alkyl benzoate) to give a much stiffer consistency than with the combinations disclosed in the prior art. In the present invention, the combination of GP-1 and Versagel S1000 also helps to constitute crystallinity to the otherwise rubbery texture of Versagel S1000 alone.

Dibutyl lauroyl glutamide is described in “The properties and Applications of N-acyl Glutamates and their Amides in Personal Care,” by J. J. Cruden et al., in Cosmetics and Toiletries Manufacture Worldwide (2002). The article describes the use of this compound as a gelling agent in mascara, lipstick, candles, and antiperspirants.

GP-1 is also known from U.S. Pat. No. 5,843,407, which also comprises a microcrystalline wax. The microcrystalline wax in the prior art tended to create opaque lipsticks. The present invention does not use microcrystalline wax, and the result is a clear and semi-clear lipstick.

Transparent lipsticks are also disclosed in U.S. Pat. No. 5,120,541, which discloses a clear stick composition having a lamellar structure that has a soap crystal growth inhibitor.

For certain applications, such as but not limited to, lipsticks used for cosmetic and personal care, it is desirable for a non-aqueous gel to have the characteristics of clearness, rigidity, low elasticity, and high pay-off. Such a gel may be used as a delivery mechanism for emollients and the like. Emollients are substances that smooth and soften the skin. There are several hundred different types of emollients, each providing its own individual texture to the skin.

C₁₂₋₁₅ alkyl benzoate is disclosed in U.S. Pat. No. 6,409,997 as a non-oil emollient compound that helps maintain the soft, smooth, and pliable appearance of the skin, but that patent discloses a high wax content, which causes the lipstick to be brittle and difficult to apply, and the gloss quality is poor.

U.S. Pat. No. 6,365,139 discloses the use of BHT as an antioxidant.

Traditionally, lipsticks have been made out of wax, oil, castor oil and a colored pigment blend. In recent years, the industry trend has been to create long-lasting lipsticks with large volumes of volatile silicone to create a thin film on the lips. The disadvantage of this technology is that the film usually becomes dry and cracked due to the escape of the volatile components. Large amounts of wax in the formulation will also make the lipstick brittle and difficult to apply, and it will negatively affect the gloss quality. Versagel® S1000 can be used to replace a portion of the wax and silicone components in a traditional lipstick formula to improve the moisturization, film formation, and texture of the finished products.

### BRIEF SUMMARY OF THE INVENTION

This invention relates generally to non-aqueous gels capable of being used as delivery mechanisms for emollients and the like. More specifically, this invention relates to non-aqueous gels possessing the properties of clarity, rigidity, low elasticity and high pay-off.

In accordance with various aspects of the invention, non-aqueous gels have been produced by utilizing thermoplastic copolymers, hydrocarbon oils, esters, and amino acid based gelling agents. The products are clear, rigid, and possess high pay-off. These properties are advantageous for use of these non-aqueous gels as delivery mechanisms for emollients and the like.

### DETAILED DESCRIPTION OF THE INVENTION

The present invention is a combination of a base material, which the inventor names “Versagel® S1000” (see Table 1) and esters and gelling agents shown in the examples set out below.

Versagel S1000 comprises approximately 80% by weight hydrogenated polyisobutene, 6% by weight butyl stearate, 6% by weight isostearyl alcohol, 1.5% by weight hydrogenated styrene/isoprene copolymers, 5% by weight. Kraton G1650® polymers, 1% by weight dibutyl lauroyl glutamide, and 0.07% by weight butylated hydroxy toluene. To make the base component of Versagel® S1000, hydrogenated polyisobutene is placed in a mixing vessel along with butylated hydroxy toluene and hydrogenated polyisobutene which is then heated to 75 C while stirring. Both polymers and dibutyl lauroyl glutamide are added to the mixture which is brought up to a range of 120-135 C, until the solids are dissolved. The temperature is then lowered and all subsequent ingredients are mixed into the composition to make the base product. The Versagel® S1000 is then used along with hydrogenated polyisobutene, C₁₂₋₁₅ Alkyl Benzoate, and dibutyl lauroyl glutamide to produce a clear or semi-clear non-aqueous gel that can be used to form a rigid lipstick with low elasticity and high pay-off. The best mode of this invention comprises 55% by weight Versagel® S1000, 25% by weight hydrogenated polyisobutene, 15% by weight C₁₂₋₁₅ Alkyl Benzoate, and 5% by weight dibutyl lauroyl glutamide.

TABLE 1
Versagel ® S1000:

| COMPO- | | | PERCENT- | |
|---|---|---|---|---|
| NENT | INCI | SUPPLIER | AGE | FUNCTION |
| Pana- | Hydrogenated | Lipo | 80.43 | Emolient |
| lane- | Polyiso- | Chemicals | | |
| L14E | butene | | | |
| Dermol | Butyl | Alzo | 6.00 | Emollient, |
| BS | Stearate | Inter- | | dermatophilic |
| | | national | | additive, |
| | | | | plasticizer |
| | | | | and solvent |
| | | | | for pigments |
| | | | | in lipsticks. |
| Tego | Isostearyl | Degussa | 6.00 | Emolient, |
| Alka- | Alcohol | | | provides the |
| nol | | | | pay-off that |
| 66 | | | | is desired |
| | | | | for the fin- |
| | | | | ished product |
| Kraton | Hydrogenated | Kraton | 1.50 | Thermoplastic |
| G1702 | styrene/ | Polymer | | elastomer |
| | isoprene | | | gellant |
| | copolymers | | | |
| Kraton | Hydrogenated | Kraton | 5.00 | Thermoplastic |
| G1650 | styrene/ | Polymers | | elastomer |
| | butadiene | | | gellant |
| | copolymers | | | |
| GP-1 | Dibutyl | Ajinomoto | 1.00 | Oil gellant; |
| | Lauroyl | Co | | in combination |
| | Glutamide | | | with Butyl |
| | | | | Stearate im- |
| | | | | proves the |
| | | | | crystallinity |
| | | | | of the gel |
| | | | | structure |
| BHT | Butylated | | 0.07 | Antioxidant |
| | Hydroxy | | | |
| | Toluene | | | |

Mixing Instructions: Measure hydrogenated polyisobutene into the mixing vessel, add BHT, and heat the oil up to 75° C. while maintaining adequate mixing. Add both polymers and GP-1 into the oil and blend the mixture at a temperature range of 120-135° C. until the solids are well dissolved. Lower the temperature to about 120° C. and add the rest of the ingredients to the mixture. Let it mix for a short while before transferring the material into storage containers.

Critical temperature for blending is 120° C. Below this temperature, the GP-1 will not fully dissolve.

The addition of the ester and alcohol will help accelerate the dissolution of GP-1. Another option of practice may be to dissolve GP-1 in the ester and alcohol blend first, then to add the mixture to the oil/polymer blend. However, this may be undesirable because it is a two-phase mixing process.

### EXAMPLE 1

| Composition | Supplier | Percentage |
|---|---|---|
| Versagel ® S1000 | Penreco | 55.00 |
| Hydrogenated Polyisobutene | Lipo | 25.00 |
| C₁₂₋₁₅ Alkyl Benzoate (ester) | Fintex | 15.00 |
| Dibutyl Lauroyl Glutamide (GP-1) | Ajinomoto | 5.00 |

Comments: The combination of ester and GP-1 gives a stiff consistency to the gel. The inventor believes that GP-1 also helps to increase the crystallinity of the otherwise rubbery texture of Versagel S1000. The result is a clear stick with good pay-off and sheen quality. The stick leaves behind a nice film and a slight tack sensation.

### EXAMPLE 2

| Composition | Supplier | Percentage |
|---|---|---|
| Versagel ® S1000 Modified | Penreco | 66.00 |
| (#1000-103) | | |
| Hydrogenated Polyisobutene | Lipo | 15.00 |
| Isostearyl Alcohol | Degussa | 3.75 |
| Butyl Stearate | Alzo | 11.25 |
| GP1 | Ajinomoto Co. | 4.00 |

Comments: The result is a much clearer stick than in Example 1, and it retained the clarity for an extended period of time. The quality of the pay-off and integrity is good.

### EXAMPLE 3

| Composition | Supplier | Percentage |
|---|---|---|
| Versagel ® S1000 | Penreco | 50.00 |
| PIB | | 25.00 |
| Finsolv TN | | 15.00 |
| Microcrystalline wax | | 5.00 |
| GP-1 | Ajinomoto Co. | 5.00 |

Comments: The composition is much less opaque, yet very stiff. Furthermore, the stick gives a nice sheen and rub off. However, the strength and transparency could be improved.

### EXAMPLE 3

| Composition | Supplier | Percentage |
|---|---|---|
| Versagel ® S1000 | Penreco | 55.00 |
| PIB | | 35.00 |
| Finsolv TN | | 5.00 |
| GP-1 | Ajinomoto Co. | 5.00 |

Comments: The stick is much softer than #1000-80.

### EXAMPLE 4

| Composition | Supplier | Percentage |
|---|---|---|
| Versagel ® S1000 | Penreco | 55.00 |
| PIB | | 20.00 |
| Finsolv TN | | 20.00 |
| GP-1 | Ajinomoto Co | 5.00 |

Comments: This is the preferred embodiment of the present invention for clear lipstick gloss.

### EXAMPLE 5

| Composition | Supplier | Percentage |
|---|---|---|
| Versagel ® S1000 | Penreco | 56.00 |
| PIB | | 25.00 |
| Finsolv TN | | 15.00 |
| GP-1 | Ajinomoto Co. | 4.00 |

Comments: This stick is about the same stiffness and clarity as Example 4.

### EXAMPLE 6

| Composition | Supplier | Percentage |
|---|---|---|
| Versagel ® S1000 | Penreco | 66.00 |
| PIB | | 15.00 |
| Finsolv TN | | 15.00 |
| GP-1 | Ajinomoto Co. | 4.00 |

Comments: This stick is more rubbery than the examples above, but it is still clear.

TABLE 2

| COMPONENTS | #838-139-01 | #838-139-02 | #838-139-04 | #838-139-07 | #838-139-08 |
|---|---|---|---|---|---|
| Hydrogenated PIB | 86.95 | 87.00 | 87.00 | 86.90 | 86.90 |
| Kraton G1702 | — | 2.60 | 0.75 | 1.50 | 1.50 |
| Kraton G1650 | 7.00 | 4.60 | 6.2 | 5.55 | 5.55 |
| DibutylLauroyl | — | 0.75 | 1.00 | 1.00 | 1.00 |
| Glutamide | | | | | |
| Isostearyl alcohol | — | — | 5.00 | — | 5.00 |
| Stearyl Alcohol | 5.00 | — | — | — | — |
| Diisopropyl | — | 5.00 | — | — | — |
| Adipate | | | | | |
| Isostearic acid | — | — | — | — | — |
| Octyl dodecanol | — | — | — | 5.00 | — |
| Butyl Stearate | — | — | — | — | — |
| C12-15 Alkyl | — | — | — | — | — |
| Benzoate | | | | | |
| BHT | 0.05 | 0.05 | 0.05 | 0.05 | — |
| Results | The finished | Translucent | Translucent | Hazy gel | Better |
| | product has better | gel, very | gel with | with good | clarity |
| | pay-off than | good pay-off | good pay-off | pay-off | with poor |
| | RE750 but is | but poor | and better | but a bit | integrity, |
| | white and Lumpy | integrity | integrity, | too tacky | very tacky |
| | | | feels tacky | | |

TABLE 3

| | #838- | #838- | #838- | #1000- |
|---|---|---|---|---|
| COMPONENTS | 139-10 | 139-16 | 139-17 | 64 |
| Hydrogenated | 79.46 | 91.5 | 83.17 | 77.43 |
| PIB | | | | |
| Kraton G1702 | 0.74 | 1.52 | 1.52 | 1.5 |
| Kraton G1650 | 5.75 | 5.93 | 5.26 | 5 |
| DibutylLauroyl | 1 | 1 | 1 | 1 |
| Glutamide | | | | |
| Isostearyl | 9 | — | 3 | 6 |
| alcohol | | | | |
| Stearyl Alcohol | — | — | — | — |
| Diisopropyl | — | — | — | — |
| Adipate | | | | |
| Isostearic acid | 4 | — | — | — |
| Octyl dodecanol | — | — | — | — |
| Butyl Stearate | — | — | 6 | 9 |
| C12-15 Alkyl | — | — | — | — |
| Benzoate | | | | |
| BHT | 0.05 | 0.05 | 0.05 | 0.07 |
| Results | Slightly | Rubbery | Translucent | Translucent |
| | hazy with | gel | gel with | gel. Clarity |
| | poor integ- | | stiff struc- | is better |
| | rity, good | | ture and | but slightly |
| | pay-off | | good pay- | softer, than |
| | | | off, | S1000 |
| | | | not tacky | |

TABLE 4

| COMPONENTS | #838-157 | #1000-54 | #1000-55 | #1000-56 | #1000-57 |
|---|---|---|---|---|---|
| Hydrogenated | 83.43 | 83.43 | 79.43 | 80.43 | 79.43 |
| PIB | | | | | |
| Kraton G1702 | 1.5 | 1.50 | 1.50 | — | 1.5 |
| Kraton G1650 | 5.0 | 5.0 | 5.0 | — | 5.0 |
| Kraton G6917 | — | — | — | — | — |
| Kraton G1651 | 2.00 | — | — | — | — |
| Kraton A6935 | — | — | — | 6.50 | — |
| Dibutyl Lauroyl | 1.00 | — | — | 1.00 | 2.00 |
| Glutamide | | | | | |
| Polyethylene | — | 1.00 | — | — | — |
| Paraffin Wax | — | — | 1.00 | — | — |
| Ozokerite Wax | — | — | 1.00 | — | — |
| Butyl Stearate | 4.00 | 6.00 | 6.00 | 6.00 | 6.00 |
| Isostearyl alcohol | 3.00 | 6.00 | 6.00 | 6.00 | 6.00 |
| BHT | 0.07 | 0.07 | 0.07 | 0.07 | 0.07 |
| Results | Clear stiff gel | Opaque soft | Opaque soft | Hazy gel with | Hazy gel that is |
| | but very | gel with no | gel with no | synerisis, | more stiff, slightly |
| | viscose for | pay-off | pay-off | rubbery, not stiff | less pay-off |
| | processing, and | | | | compared to S1000 |
| | not enough | | | | |
| | pay-off | | | | |

* Kraton G1651 is a higher MW tri-block polymer.
* Kraton A6935 is a polymer resembling G1651, but with styrene in the middle block to improve compatibility with polar ingredients.

TABLE 5

| COMPONENTS | #1000-58 | #1000-59 | #1000-65 | #1000-60 | #1000-103A |
|---|---|---|---|---|---|
| Hydrogenated | 78.43 | 76.43 | 77.18 | 71.43 | 83.43 |
| PIB | | | | | |
| Kraton G1702 | 1.50 | 1.50 | 1.50 | 1.50 | 1.50 |
| Kraton G1650 | 5.00 | 5.00 | 5.00 | 5.00 | 5.00 |
| Kraton G6917 | — | — | — | — | — |
| Kraton G1651 | — | — | — | — | — |
| Kraton A6935 | — | — | — | — | — |
| Dibutyl Lauroyl | 3.00 | 5.00 | 1.25 | 10.00 | 1.00 |
| Glutamide | | | | | |
| Polyethylene | — | — | — | — | — |
| Paraffin Wax | — | — | — | — | — |
| Ozokerite Wax | — | — | — | — | — |
| Butyl Stearate | 6.00 | 6.00 | 9.00 | 6.00 | 9.60 |
| Isostearyl alcohol | 6.00 | 6.00 | 6.00 | 6.00 | 2.40 |
| BHT | 0.07 | 0.07 | 0.07 | 0.07 | 0.07 |
| Results | Hazy gel | Opaque gel, | Translucent | Opaque gel, | The gel has much |
| | but very | very stiff, | gel with good | extremely | better clarity and |
| | stiff, less | almost | pay-off, | stiff, no pay- | comparable stiffness |
| | pay-off | resembles a | comparable | off | to S1000 |
| | compared | wax blend, | stiffness to S- | | |
| | to S1000 | loosing the | 1000 | | |
| | | pay-off | | | |

* Kraton G1651 is a higher MW tri-block polymer.
* Kraton A6935 is a polymer resembling G1651, but with styrene in the middle block to improve compatibility with polar ingredients.

TABLE 6

| COMPONENTS | SUPPLIER | #1000-68 | #1000-70 |
|---|---|---|---|
| Ozokerite Wax | (Strahl&Pitsch) | 15.00 | 15.00 |
| Beeswax | (Strahl&Pitsch) | 5.00 | 5.00 |
| Polyethylene wax | (Allied Signal) | 4.00 | 6.00 |
| Polybutene H300 | (Lipo) | 4.50 | — |
| Hydroxylated | (Rita) | 2.50 | 2.50 |
| Lanolin | | | |
| Octyl Dodecanol | (Henkel) | 11.50 | 11.50 |
| Phenyl | (Dow Corning) | 2.00 | 2.00 |
| Trimethicone | | | |
| Cyclomethicone | (Dow Corning) | 40.70 | 25.00 |
| Versagel S1000 | (Penreco) | — | 22.75 |
| Parabens | (Tri-K) | 0.25 | 0.25 |
| Mica | | 10.00 | 5.00 |
| BHT | | 0.05 | 0.05 |
| C33-134 Balck | (Sun Chemical) | 0.25 | 0.25 |
| Iron Oxide | | | |
| C33-8075 Red Iron | (Sun Chemical) | 0.50 | 0.50 |
| oxide | | | |
| C47-056 Titanium | (Sun Chemical) | 1.50 | 1.50 |
| Oxide | | | |
| C33-8073 Yellow | (Sun Chemical) | 0.50 | 0.50 |
| Iron Oxide | | | |
| C70-5270 FD&C | (Sun Chemical) | 0.50 | 0.50 |
| Yellow 6 Al Lake | | | |
| C19-011 D&C Red | (Sun Chemical) | 1.25 | 1.25 |
| 7 ca lake | | | |
| Obersevations | | Finished | Finished |
| | | product | product |
| | | feels dry | feels rich |
| | | with poor | with a lot |
| | | tack | of body, |
| | | | some tack |

The purpose of these examples is to teach those skilled in the art of cosmetic chemistry the component ranges of the present invention. These examples indicate that the combination of Kraton G1702 and G1650 and GP-1 gives the best matrix for a translucent solid gel, butyl stearate helps strengthen the gel structure and solubilize the GP-1, and isostearyl alcohol reduces tackiness and improves pay-off.

Although the inventor has described specific embodiments of his invention in this specification, these are only illustrative of the invention. The scope of the invention should be limited only by the appended claims and their legal equivalents.

1. A non-aqueous gel composition comprising at least one anhydrous gel, at least one polymer, at least one diblock copolymer, at least one ester, and at least one gelling agent, wherein said at least one anhydrous gel comprises at least one emollient, at least one thermoplastic elastomer gellant, at least one oil gellant, and at least one antioxidant.

2. A composition of claim 1, wherein said at least one polymer is hydrogenated polyisobutene.

3. A composition of claim 1, wherein said at least one ester is C₁₂-C₁₅ Alkyl Benzoate.

4. A composition of claim 1, wherein said at least one gelling agent is dibutyl lauroyl glutamide.

5. A non-aqueous gel composition comprising at least one anhydrous gel, at least one polymer, at least one ester, and at least one gelling agent, wherein said at least one anhydrous gel comprises at least one emollient, at least one thermoplastic elastomer gellant, at least one oil gellant, and at least one antioxidant

6. A composition of claim 5, wherein said at least one emollient is hydrogenated polyisobutene.

7. A composition of claim 5, wherein said at least one emollient is butyl stearate.

8. A composition of claim 5, wherein said at least one emollient is isostearyl alcohol.

9. A composition of claim 5, wherein said at least one thermoplastic elastomer gellant is hydrogenated styrene/isoprene copolymers.

10. A composition of claim 5, wherein said at least one thermoplastic elastomer gellant is hydrogenated styrene/butadiene copolymers.

11. A composition of claim 5, wherein said at least one oil gellant is dibutyl lauroyl glutamide.

12. A composition of claim 5, wherein said at least one antioxidant is butylated hydroxyl toluene.

13. A composition of claim 5, wherein said anhydrous gel is present in the amount ranging from about 47% to about 62% by weight relative to the total weight of the composition.

14. A non-aqueous gel composition comprising at least one anhydrous gel, at least one polymer, at least one ester, and at least one gelling agent, wherein said at least one polymer is hydrogenated polyisobutene and is present in the amount ranging from about 17% to about 32% by weight relative to the total weight of the composition.

15. A composition of claim 14, wherein said polymer is present in the amount ranging from about 17% to about 32% by weight relative to the total weight of the composition.

16. A non-aqueous gel composition comprising at least one anhydrous gel, at least one polymer, at least one ester, and at least one gelling agent, wherein said at least one ester is C₁₂-C₁₅ Alkyl Benzoate and is present in the amount ranging from about 8% to about 22% by weight relative to the total weight of the composition.

17. A composition of claim 16, wherein said ester is present in the amount ranging from about 8% to about 22% by weight relative to the total weight of the composition.

18. A non-aqueous gel composition comprising at least one anhydrous gel, at least one polymer, at least one ester, and at least one gelling agent, wherein said at least one gelling agent is dibutyl lauroyl glutamide.

19. A composition of claim 18, wherein said gelling agent is present in the amount ranging from about 0.001% to about 15% by weight relative to the total weight of the composition.

20. The composition of claim 1, wherein said at least one polymer is hydrogenated polyisobutene, said at least one ester is C12-C15 Alkyl benzoate, and said at least one gelling agent is dibutyl lauroyl glutamide.

21. The composition of claim 14, wherein said at least one anhydrous gel comprises at least one emollient, at least one thermoplastic elastomer gellant, at least one oil gellant, and at least one antioxidant.

22. The composition of claim 16, wherein said at least one anhydrous gel comprises at least one emollient, at least one thermoplastic elastomer gellant, at least one oil gellant, and at least one antioxidant.

23. The composition of claim 18, wherein said one anhydrous gel comprises at least one emollient, at least one thermoplastic elastomer gellant, at least one oil gellant, and at least one antioxidant.
